(19) United States
(12) Patent Application Publication
(10) Pub. No.: US 2025/0228502 A1
(43) Pub. Date: Jul. 17, 2025

(54) AGENTIC GPT-BASED INTERACTIVE ELECTROCARDIOGRAPHIC ANALYSIS

(71) Applicant: CardiacCloud AI, Inc., Santa Clara, CA (US)

(72) Inventors: Sreeram Pydah, Livermore, CA (US); Sujoya Dey, Tiburon, CA (US); Nelson Jack Mangione, Brentwood, TN (US); Ravi Illango, Cupertino, CA (US); Shashi Ranjan, Stretton (AU)

(21) Appl. No.: 19/017,986

(22) Filed: Jan. 13, 2025

Related U.S. Application Data
(60) Provisional application No. 63/620,226, filed on Jan. 12, 2024.

(51) Int. Cl. A61B 5/00; A61B 5/024; A61B 5/352; A61B 5/358; A61B 5/36; A61B 5/363; G06F 16/2452; G16H 10/60

(52) U.S. Cl. CPC A61B 5/7282; A61B 5/02405; A61B 5/352; A61B 5/358; A61B 5/36; A61B 5/363; A61B 5/742; A61B 5/746; A61B 5/7475; G06F 16/24522; G16H 10/60

(57) ABSTRACT

A system for interactive ECG monitoring is described. The system includes a data repository storing pre-processed ECG data. The pre-processed ECG data is associated with historical data, real-time data, or both derived from a plurality of ECG recorders. The pre-processed ECG data includes ECG measurements extracted or derived from raw ECG signals and annotations of cardiac events. Further, the system includes a multi-agent query processor to receive and process an input message related to health of a subject, retrieve relevant data elements from the pre-processed ECG data, raw ECG signals, or both based on the processed input message, compute metrics corresponding to the input message based on the retrieved data elements, and generate a response to the input message using an LLM or at least one agent to integrate retrieved data elements and computed metrics. The response is presented on a user interface to a healthcare provider.

### CROSS REFERENCE TO RELATED APPLICATIONS

This patent application claims the benefit of and priority to U.S. Provisional Patent Application Ser. No. 63/620,226, filed on Jan. 12, 2024, titled “GPT-based interactive electrocardiographic monitoring studies including ambulatory cardiac monitoring studies for diagnosing cardiac conditions” the disclosure of which is hereby incorporated by references for all purposes.

### TECHNICAL FIELD

This application relates to the field of interactive electrocardiographic (ECG) analysis and, more specifically, to a system and method for interactively analyzing pre-processed ECG data using a large language model (LLM).

### BACKGROUND

Over the years, advancements in cardiac monitoring technologies have significantly enhanced the ability to collect and analyze electrocardiographic (ECG) data. Traditional methods like Holter monitors and Event recorders, along with newer technologies such as mobile telemetry, loop recorders, and wearable devices, have become indispensable tools for continuous cardiac monitoring. These devices generate vast amounts of ECG data, capturing a patient's heart activity over extended periods, enabling the detection of critical cardiac events including arrhythmias, pauses, conduction abnormalities, ischemia, and other significant cardiac occurrences.

However, this progress has been accompanied by significant challenges in effectively interpreting the collected data due to data volume, noise, artifacts, and the need for accurate detection of subtle cardiac abnormalities. These challenges underscore the need for innovative solutions that can efficiently and accurately analyze large volumes of ECG data, identify critical events, and provide clinicians with actionable insights.

### BRIEF DESCRIPTION OF THE DRAWINGS

FIG. 1 illustrates an example system, depicting an agentic GPT-based interactive ECG analysis platform to interactively analyze ECG data and generate responses based on user queries;

FIG. 2 is an example sequence diagram illustrating a sequence of events to interactively analyze ECG data and generate responses based on user queries;

FIG. 3 is a block diagram of the agentic GPT-based interactive ECG analysis platform of FIG. 1, illustrating a data flow between the components;

FIG. 4 is a flow diagram illustrating an example method for analyzing pre-processed ECG data;

FIG. 5 is an example ECG waveform, depicting the ECG measurements in the context of the present invention;

FIG. 6A is a schematic diagram, depicting an example interaction between a physician and a chatbot;

FIG. 6B is an example user interface, depicting an example interaction between a physician and a chatbot; and

FIG. 7 is a block diagram of an example computing device including non-transitory computer-readable storage medium storing instructions to analyze pre-processed ECG data.

The drawings described herein are for illustrative purposes and are not intended to limit the scope of the present subject matter in any way.

### DETAILED DESCRIPTION

Examples described herein may provide an enhanced computer-based method, technique, and system to interactively analyze electrocardiographic (ECG) data from various sources and generate insights to enhance the diagnosis of cardiovascular diseases. The paragraphs [0012] to [0017] present an overview of the Electrocardiographic (ECG) studies, existing methods to analyze ECG data, and drawbacks associated with the existing methods.

ECG studies are crucial for the diagnosis of various cardiac events, such as arrhythmias, pauses, conduction abnormalities, ischemia and the like. An ECG is a primary physiological measurement used for assessing the cardiac health of a patient. Cardiac monitoring devices such as Holter monitors, event recorders, loop recorders, and wearable devices, can generate large volumes of ECG data that capture a patient's heart activity over extended periods, enabling the detection of critical cardiac events.

However, effectively interpreting the growing volume of ECG data presents significant challenges. These challenges include:
1. Data Volume: The sheer volume of data generated by modern cardiac monitoring devices presents a significant analytical burden for clinicians. In addition, when you amalgamate data from multiple electrocardiographic recordings, i.e., Holters, Loop Recorders, Stress test, etc., conducted over a period of time, the complexity of data and the sheer volume is much more pronounced.
2. Data Noise and Artifacts: ECG signals are often contaminated by noise and artifacts arising from various sources, including patient movement, electrode placement, and electrical interference, hindering accurate data interpretation.
3. Detection of Subtle Abnormalities: Many clinically significant cardiac events, such as subtle arrhythmias or early signs of ischemia, can be difficult to detect manually, requiring expertise and careful analysis.

In some existing methods, a substantial body of research has focused on addressing these challenges through signal preprocessing. Techniques to filter noise, such as baseline wander removal, muscle noise suppression, and power line interference mitigation, are well-established in ECG signal processing. These preprocessing methods are crucial for ensuring the quality and reliability of the ECG data, particularly for accurate detection of arrhythmias or deviations in key cardiac markers. Advanced feature extraction methods have also been developed to derive clinically relevant metrics from ECG signals. These metrics, such as R-R intervals, heart rate variability, QT intervals, ST-segment deviations, and PQ intervals, provide deeper insights into a patient's cardiac function. Studies have shown that R-R variability, for instance, is a strong indicator of autonomic nervous system regulation, while prolonged QT intervals are associated with an increased risk of arrhythmias.

In other existing methods, automated annotation systems represent another significant advancement in the field. Machine learning and deep learning models have been developed to identify and classify arrhythmic events from the ECG data. These models are capable of detecting patterns in raw waveforms and tagging events such as atrial fibrillation, ventricular tachycardia, bradycardia, pauses, and heart blocks. While these systems demonstrate high efficiency, their accuracy can be affected by overlapping waveforms, borderline measurements, or noise within the data. To mitigate these limitations, several studies have emphasized the importance of combining automated detection with expert human review. A human-in-the-loop approach has been shown to enhance the reliability of ECG annotations by leveraging clinical expertise to validate or correct machine-generated outputs.

In other existing approaches, centralized data repositories for ECG data storage and management have also been the subject of considerable research. Structured storage solutions that integrate raw ECG data, derived features, and annotations with metadata such as timestamps and patient identifiers have become standard. These repositories enable efficient data retrieval and analysis, supporting both real-time applications and longitudinal studies. Furthermore, research has demonstrated the utility of combining real-time and historical data for trend analysis and risk prediction. For example, studies have shown that time-series analysis of QT intervals or ST-segment deviations can help clinicians detect subtle changes in cardiac health, enabling early detection and intervention.

Existing research has laid a strong foundation for leveraging ECG data for advanced clinical applications. By combining high-quality signal preprocessing, feature extraction, expert annotation, and structured storage, current systems have significantly improved the reliability and accuracy of cardiac monitoring. These advancements have paved the way for emerging technologies, including artificial intelligence, machine learning, and Generative AI.

Examples described herein may provide a Generative Pre-trained Transformer (GPT)-based interactive ECG monitoring system for analyzing pre-processed ECG data. The system receives an input message regarding health of a subject and processes the input message to identify data elements from the pre-processed ECG data. The pre-processed ECG data is associated with historical data, real-time data, or both derived from a plurality of electrographic recorders. Also, the pre-processed ECG data includes ECG measurements extracted or derived from raw ECG signals and annotations of cardiac events. Further, the system retrieves the identified data elements from a data repository, computes metrics corresponding to the input message based on the retrieved data elements, and generates a response to the input message using a LLM or an agent by integrating the retrieved data elements. Furthermore, the system presents the response to the healthcare provider through a user interface.

Examples described herein may augment GPT models with specialized agents such as SQL agent, ECG strip agent, analysis agent, and insights agent to analyze data from various sources, including Standard 12-lead ECGs, Holter monitors, mobile cardiac telemetry, cardiac event monitors, stress tests, electrophysiology studies, implanted loop recorders, pacemakers, defibrillators, and wearables over time. Techniques utilizing GPT models and multi-agent systems significantly enhance the diagnosis of cardiovascular diseases when applied to ECG monitoring, including ambulatory cardiac monitoring.

The system dynamically responds to physician queries by providing both textual and graphical information, significantly aiding in the detection of cardiovascular diseases, such as arrhythmias. The system offers interactive visualizations related to arrhythmias, heart rate trends, symptom correlations, morphology, segment analysis, and the like. The system can process both real-time and recorded ECG data to provide an interactive dashboard for physicians and healthcare providers, enhancing their ability to analyze and diagnose cardiovascular conditions. This dashboard not only answers queries but also provides valuable insights, identifies early signs of disease progression, employs predictive analytics, and offers actionable recommendations. Further, the GPT-driven, multi-agent capabilities create a robust framework for advancing cardiac diagnostics, enabling early detection, facilitating preventive care, and ultimately improving patient outcomes, and reducing the burden of cardiovascular diseases.

Thus, the GPT-based interactive ECG monitoring system revolutionizes the ECG data analysis by seamlessly integrating key elements into a scalable and robust platform. Unlike many existing solutions that address specific components of the ECG analysis pipeline in isolation, our system offers a holistic approach, delivering actionable insights for healthcare providers.

In the following description, for purposes of explanation, numerous specific details are set forth in order to provide a thorough understanding of the present techniques. However, the example apparatuses, devices, and systems, may be practiced without these specific details. Reference in the specification to “an example” or similar language means that a particular feature, structure, or characteristic described may be included in at least that one example but may not be in other examples.

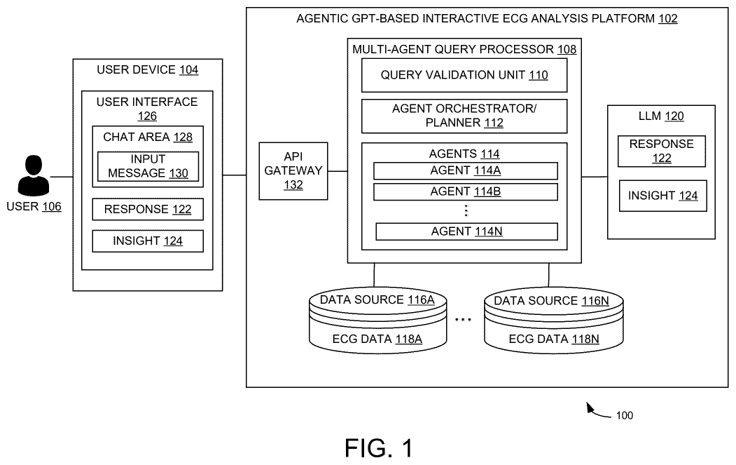

Referring now to the figures, FIG. 1 illustrates an example system 100, depicting an agentic GPT-based interactive ECG analysis platform 102 to interactively analyze ECG data (e.g., data 118A-118N) and generate responses based on user queries (e.g., 130). System 100 may include the agentic GPT-based interactive ECG analysis platform 102 and a user device 104 communicatively coupled to the agentic GPT-based interactive ECG analysis platform 102.

Further, agentic GPT-based interactive ECG analysis platform 102 includes a data repository (e.g., data sources 116A-116N) storing pre-processed ECG data. The pre-processed ECG data may include cardiac features extracted/computed/derived from raw ECG signals and annotations of cardiac events. In some examples, the data repository also includes raw signals. Furthermore, agentic GPT-based interactive ECG analysis platform 102 may include a multi-agent query processor 108 configured to interact with the data sources 116A-116N and large language models (LLMs) 120.

During operation, multi-agent query processor 108 receives and processes an input message related to health of a subject, retrieves relevant data elements from the pre-processed ECG data (e.g., 118A-118N), raw ECG signals, or both based on the processed input message 130. In an example, multi-agent query processor 108 may include a validation unit 110 to validate the input message 130 based on predefined rules and data schemas prior to processing the input message 130. Further, multi-agent query processor 108 computes metrics corresponding to the input message 130 based on the retrieved data elements and generates a response 122 and/or the insight 124 to the input message 130 using an LLM 120 or at least one agent 114 to integrate the retrieved data elements and the computed metrics. Furthermore, multi-agent query processor 108 may present the response 122 and/or the insight 124 generated by the LLM 120 to a healthcare provider (e.g., a user 106) via a user interface 126 of the user device 104.

In an example, multi-agent query processor 108 may perform semantic analysis on the received input message 130 to determine an intent of the input message and to identify data or visualizations required to fulfil the intent. Further, multi-agent query processor 108 may select at least one agent (e.g., agents 114A-114N) based on the identified data or visualizations required to fulfil the intent of the input message. When more than one agent is selected, multi-agent query processor 108 comprises an agent orchestrator/planner 112 to coordinate the execution of the selected plurality of agents 114 in a predetermined sequence. Further, multi-agent query processor 108 may receive outputs from each of the selected plurality of agents 114, where the outputs include the identified data elements and/or the computed metrics. Also, when more than one agent is selected, multi-agent query processor 108 aggregates the received outputs from the selected plurality of agents 114 into a unified response. The unified response is then displayed on user interface 126.

For example, consider that the multi-agent query processor 108 may select an SQL agent, an insights agent, and an ECG strip agent based on the identified data or visualizations required to fulfil the intent of the input message 130. In this example, the SQL agent may retrieve structured data from the data repository. Further, the insights agent may derive and compute contextual insights related to the input message 130. Furthermore, the ECG strip agent may generate visual representations of the retrieved ECG data. Further, the agent orchestrator/planner 112 coordinates the execution of the SQL and ECG agents to generate the response 122 and/or insights 124. In the following paragraphs, FIG. 1 is explained in detail with examples.

The agentic GPT-based interactive ECG analysis platform 102 integrates data sources 116A-116N (where N is a positive integer) of ECG data 118A-118N for use with agentic GPT-based interactive ECG analysis platform 102. For example, ECG data 118A-118N may be obtained from various cardiac monitoring devices such as Standard 12-lead ECG, Holter monitors, mobile cardiac telemetry, cardiac event monitoring, stress tests, electrophysiology studies, implanted loop recorders, pacemakers, defibrillators, and wearables over time.

Cardiac monitoring devices gather the ECG data 118A-118N at varying resolutions and sampling frequencies. To ensure data quality, preprocessing is crucial for cleaning and refining the raw ECG signals. This preprocessing pipeline involves applying advanced filtering algorithms to remove baseline wander, noise, and power line interference while preserving essential waveform characteristics such as R-waves and ST-segments. Specialized pattern recognition methods are then employed to identify and exclude square-wave artifacts and other non-informative segments, ensuring only meaningful data is retained. This step not only improves the reliability of feature extraction but also enhances the accuracy of subsequent annotations and analyses.

Beyond artifact removal, preprocessing extracts ECG measurements or cardiac features such as R-R intervals, QT intervals, ST-segment deviations, and PQ intervals. These features are crucial for assessing heart rate variability, arrhythmic risks, and conduction anomalies. These features are meticulously calculated and stored alongside the raw data, forming a comprehensive dataset enriched with metadata such as timestamps, patient identifiers, and device details. This structured tagging facilitates event categorization and longitudinal analysis, enabling actionable insights for treatment optimization.

For example, trends in R-R variability can indicate autonomic dysfunction, while prolonged QT intervals, potentially correlated with medication adjustments, provide critical insights into arrhythmic risks. Similarly, persistent ST-segment deviations over time may signal ischemic episodes requiring immediate clinical attention. These features, combined with their corresponding metadata, create a multi-dimensional view of the patient's cardiac profile, enabling both high-level summaries and detailed examinations.

Following preprocessing, the dataset undergoes annotation to classify significant cardiac events. Advanced algorithms can detect arrhythmias (e.g., tachycardia, bradycardia, and pauses), heart blocks, and compute deviations in derived metrics. Human review ensures accuracy by correcting discrepancies in automated tagging. For instance, flagged QT intervals or ST deviations are validated by trained technicians to account for noise or borderline values, capturing subtle nuances crucial for clinical reliability. This dual-layered approach ensures precise and clinically robust annotations.

Following preprocessing and annotation, the dataset is stored in a secure repository (e.g., data sources 116A-116N) designed to handle large volumes of data with redundancy and scalability. Each dataset 118A-118N includes raw ECG signals, preprocessed features, and verified annotations, all linked through structured metadata. This enables seamless integration with real-time and historical records. For instance, a record might include average R-R intervals, maximum QT intervals, instances of ST-elevation, and timestamps of annotated events, providing clinicians with a comprehensive cardiac profile.

Further, the structured dataset 118A-118N supports advanced analyses, such as time-series evaluations and arrhythmia trend detection, by leveraging derived features like R-R intervals, QT intervals, and PQ intervals. For example, increasing R-R variability might suggest autonomic nervous system dysfunction, while consistent PQ prolongation could indicate progressive conduction block. By identifying these trends early, clinicians can intervene promptly, potentially improving patient outcomes.

Furthermore, the integration of annotated data with real-time and historical records supports longitudinal studies, enabling comparisons over extended periods. For example, analyzing QT interval trends across monitoring sessions can reveal a patient's response to medication, while R-R variability trends can indicate the onset of arrhythmia. Contextual correlations, such as those with activity levels or clinical observations, provide deeper insights into the patient's condition.

The preprocessing pipeline prepares data for downstream applications, ensuring reliability and informativeness. By removing artifacts, extracting meaningful features, and validating annotations through expert review, the system produces a high-quality dataset optimized for clinical decision-making. This comprehensive approach enhances the effectiveness of advanced systems like agentic GPT-based interactive ECG analysis platform 102, which utilizes large language models (LLMs) 120 and agents 114 to interact with healthcare providers and deliver accurate insights, enabling personalized and effective cardiac care.

The workflow begins with annotated and preprocessed ECG data 118A-118N being passed to the agentic GPT-based interactive ECG analysis platform 102 for advanced query processing and analysis. This dataset 118A-118N includes detailed annotations of significant cardiac events, such as arrhythmias, pauses, tachycardias, and other irregularities, as well as precomputed metrics like heart rate variability, R-R intervals, QT intervals, and ST-segment deviations. The annotations, initially generated by automated algorithms, are reviewed and validated by cardiac technicians to ensure clinical accuracy and reliability. By integrating preprocessed and annotated data, the system 100 provides the GPT-based interactive ECG analysis platform 102, which includes the LLM 120, with a comprehensive and high-quality foundation. This enables the LLM 120 to deliver precise, contextually relevant, and actionable insights in response to physician queries or input messages 130.

The workflow progresses when a cardiologist (i.e., user 106) inputs a query or input message 130 through a chatbot interface (e.g., a chat area 128). Users 106 can ask detailed questions, such as requesting specific patient data, retrieving ECG strips, or seeking insights into monitoring results. This flexibility extends to queries that involve studies from multiple patients, or multiple studies of a single patient, or a single patient study as shown in below examples:
4. Multi-Patient Study Queries: A physician can ask, “Provide an analysis of arrhythmia trends for all my patients in the age group of 75 to 80 years for studies conducted in the last 12 months.
5. Multi-Study for a Single Patient Queries: The system can also handle questions like, “What is the variation in Atrial Fibrillation across the last three studies for patient with MRN 4701? I would like to see the differences in frequency, duration, and burden. This enables physicians to explore trends over time and across various studies for a single patient, giving them the ability to determine the deterioration, improvement, or existence of a cardiovascular disease.
6. Single-Patient Queries: Today, most of these studies provide an interim report followed by a final End-of-Study report. The physician does not have access to each and every heartbeat or, for that matter, each and every episode. With our ‘GPT-based interactive ECG monitoring system,’ physicians will have the ability to ask for information like “Show me 8-second strips of all AFib onsets on day 2 of the study”, “Show me 6-second strips of all episodes when the patient experienced dizziness”, or “What was the night-time average heart rate?”, and the like.

When the user 106 submits an input message or query 130, the chatbot collects the input and formats it into an application programming interface (API) call, which is then sent to an API Gateway 132. Acting as the central entry point for backend processing, the API Gateway 132 validates the input message 130 for completeness. The API gateway may check for required fields such as valid user IDs, study identifiers, patient IDs, and the like. If any critical information is missing or invalid, the system generates an appropriate error response (e.g., “Error: User ID missing or invalid”) and immediately returns it to the chatbot interface to notify the physician, ensuring clear and smooth communication.

For queries that pass validation, the API Gateway 132 forwards the request to the multi-agent query processor also referred to as the multi-agent orchestrator/planner 112, a core component designed to coordinate multiple specialized agents 114. Some example specialized agents 114 may include:
7. SQL Agent: This agent converts the physician's query into an executable SQL statement and retrieves contextual data from the database, such as patient metrics, annotations, and longitudinal trends. It enables the system to process queries like:
   “Retrieve the average heart rate and arrhythmia trends for patient 4701 over the last three weeks.”
   “What are the most common arrhythmias across all my patients older than 80 years?”
8. ECG Strip Agent: In case of visualization requests or queries, this agent takes the data retrieved by the SQL agent and generates visual representations of raw ECG data for detailed review. It allows the physician to measure parameters graphically as well. It handles queries that require graphical insights, such as:
   “Show the ECG strips for all detected arrhythmias in the past 24 hours for patient 4701.”
   “Provide ECG visualizations for all the AFibs experienced by patient 4701 in the last three studies.”
9. Insights Agent: The chatbot includes an ‘insights window’ that contextually generates information around the query/information requested by the healthcare provider. For example, if a healthcare provider posed a question “What is the maximum heart rate for patient 4701 ?”, the chatbot would respond with “The maximum heart rate was 176 bpm on Day 2 at 7:48 pm.” However, if the insights window/section were enabled, the Insights agent would display the following information:
   “The total study was 3 days long. Maximum heart rate during nighttime was 126 bpm. Maximum heart rate on Day 1 was 148 bpm at 8:20 pm. Max heart rate on Day 3 was 156 bpm at 6:50 pm.”
10. Other Specialized Agents: The system's architecture allows for the seamless integration of additional agents to meet evolving clinical needs and as a natural evolution of the system. Some of the other agents include:
   Report Generation Agent: Creates summarized or detailed PDF reports based on the queries and query results. This agent will allow the healthcare provider to add more reports to a published report, etc.
   Data Validation Agent: Validates the integrity and accuracy of retrieved data before it is presented to the physician/healthcare provider.
   Alerting Agent: Monitors real-time data for critical events, such as prolonged pauses or high-risk arrhythmias, and generates alerts along with insights for immediate action.

The multi-agent query processor 108 is the core of the agentic GPT-based interactive ECG analysis platform 102. When a query is received, the multi-agent query processor 108 performs the following tasks:
11. Semantic Analysis: Breaks down the query to understand its intent and the required data or visualizations.
12. Agent Selection: Identifies which of the agents 114 are needed to fulfill the query. For example, a query requiring both raw data and visual ECG strips would trigger the SQL agent and the ECG strip agent.
13. Coordination and Execution: Ensures agents 114 execute their tasks in the correct sequence. For instance, retrieving ECG data from the database along with annotations (i.e., the SQL agent) precedes the visual presentation of the annotated strips (i.e., the ECG strip agent).
14. Response Aggregation: Consolidates outputs from multiple agents 114 into a unified response, ensuring the information is clear and actionable for the physician.

Thus, the examples described herein may allow the system 100 to process even the most complex queries by breaking them into manageable tasks and leveraging the specific strengths of each agent 114A-114N. This design ensures flexibility, scalability, and the ability to handle queries involving studies from multiple patients and multiple studies of a single patient. Once the API Gateway 132 forwards the query to the multi-agent query processor 108, the multi-agent query processor 108 interprets the query, performs semantic analysis, and determines which agents to invoke. For instance:
15. If the query involves patient metrics over a time period, the SQL agent is activated to retrieve structured data such as heart rate variability, arrhythmia frequencies, or R-R intervals.
16. If the query requires visual ECG data, the ECG strip agent is invoked to generate annotated ECG segments for specific events.

This workflow ensures that physicians can receive detailed, accurate, and contextually relevant insights, regardless of the complexity or scope of their queries. The inclusion of multi-patient and multi-study support significantly enhances the system's utility, empowering physicians to make informed, data-driven decisions to effectively diagnose cardiovascular diseases. By enabling comprehensive analysis across patients and studies, the system 100 offers a more dynamic and interactive approach to cardiac care, ultimately improving patient outcomes.

In some examples, the system 100 supports both multi-patient queries and multi-study analyses for individual patients, enabling physicians to compare data across different studies conducted for the same patient or analyze trends across multiple patients. For example:
17. A query like “What are the arrhythmia trends for patient “X” over their last three studies?” retrieves and consolidates results across multiple datasets for that patient.
18. A broader query such as “Compare average heart rates of all my patients over 80 years” retrieves aggregated metrics for multi-patient analysis.

Upon receiving the query, the multi-agent query processor 108 interprets it, performs semantic analysis, and decides which agents to invoke. For instance:
19. A query requesting “What are the arrhythmia trends over the past 48 hours?” triggers the SQL agent to retrieve annotated arrhythmia data and associated metrics.
20. A query like “Show the ECG strips for all detected arrhythmias in the past 24 hours” activates the ECG strip agent, which generates ECG segments annotated with arrhythmic events.

For example, the SQL agent interacts with a centralized repository (e.g., data sources 116A-116N) that contains both historical and real-time data. This repository includes structured annotations, raw ECG signals, and processed metrics, all tagged with patient IDs, timestamps, device types, and the like to enable precise data retrieval. For example, a query about arrhythmia trends over the past 48 hours retrieves both annotated arrhythmias and associated metrics like heart rate variability, enabling comprehensive responses. Historical data is referenced to highlight long-term changes in cardiac patterns, helping physicians detect potential risks or improvements in the patient's condition.

The ECG strip agent is selected when visual aids are required. For instance, if a cardiologist queries, “Show the top three arrhythmias observed for patient “X” in the past 48 hours, along with ECG strips,” the system activates the ECG agent to extract and render annotated ECG strips for the top arrhythmias, such as:
21. Sinus Bradycardia (4.6%)—episodes of slow heart rhythm.
22. Sinus Tachycardia (1.2%)—episodes of elevated heart rate potentially linked to stress or activity.
23. Pause (0.2%)—brief interruptions in electrical activity that may warrant further investigation. The strips are then provided alongside the query results, enhancing the physician's understanding with visual evidence.

Further, the system 100 integrates prompt engineering to optimize query processing. This involves refining the input query to ensure that the LLM 120 or the multi-agent query processor interprets its intent, whether the physician is asking for specific metrics (e.g., average heart rate), annotated events, or broader insights like longitudinal cardiac trends. This ensures the LLM 120 generates precise, context-aware responses tailored to cardiologists' needs.

After the agents 114A-114N process the query, the results are synthesized by the LLM 120 into a structured and actionable response. For instance, if a physician queries, “What is the patient's average heart rate during nighttime hours over the past week?”, the SQL agent (e.g., 114A) retrieves the relevant metrics, and the LLM 120 formats the response into a summary with clinical significance. Similarly, if a physician requests a comparative analysis of arrhythmias across two periods, the SQL agent retrieves the data, and the ECG strip agent (e.g., 114B) provides visual comparisons. The LLM 120 then synthesizes these results into a comprehensive and insightful response for the physician.

The workflow described herein may support both historical and real-time data streams. For example, a query such as “What is the current heart rate and any recent arrhythmias for patient “X” ?” involves the SQL agent accessing live data, while the ECG strip agent generates visualizations of recent events. The system 100 responds with real-time insights, such as “Patient X's current heart rate is 78 bpm, and no arrhythmias have been detected in the past 30 minutes,” enabling timely clinical interventions.

The multi-agent query processor 108 excels at synthesizing complex datasets into conversational, medically accurate responses. By combining annotated ECG data, preprocessed metrics, and advanced prompt engineering, the system 100 offers a holistic view of the patient's cardiac health. Physicians can query details such as “List all instances of supraventricular tachycardia detected within the last 48 hours, along with ECG strips,” and receive both textual summaries and visual aids. This capability transforms how physicians' access and interpret ECG data, making it an interactive and dynamic resource rather than a static report.

Thus, the system's chatbot interface minimizes the learning curve, enabling physicians to focus on clinical insights rather than software mechanics. By integrating advanced natural language processing, multi-agent coordination, and real-time interaction, the system 100 ensures robust and patient-centered cardiac care. This holistic approach empowers physicians to make informed, data-driven decisions, ultimately leading to improved patient care and outcomes.

During operation, the multi-agent query processor 108 preprocesses and transforms user queries 130 received from the user 106, retrieves relevant document or data from available data sources 116A-116N by invoking appropriate agents 114, and generates responses 122 and/or insights 124 to the user queries 130. The agents 114 generates responses 122 and/or insights 124 to the input messages 130 directly, either from the LLM 120 or using the knowledge representations available to the agents 114, to serve user intent of the input messages 130. In an example, the multi-agent query processor 108 uses a single LLM 120 to generates the responses 122 and/or insights 124 to the input queries 130. In another example, the multi-agent query processor 108 uses a plurality of LLMs 120 to generate the responses 122 and/or insights 124 to the input queries 130.

Furthermore, the agentic GPT-based interactive ECG analysis platform 102 is accessible to a user 106 using a user interface 126 on a user device 104 of the user 106. In some examples, a plurality of devices 104 are in communication with the agentic GPT-based interactive ECG analysis platform 102 and a plurality of users 104 have access to the agentic GPT-based interactive ECG analysis platform 102. In some examples, the agentic GPT-based interactive ECG analysis platform 102 is on a server (e.g., a cloud server) remote from the user device 104 of the user 106 accessed via a network. The network may include the Internet or other data link that enables transport of electronic data between respective devices and/or components of the system 100. For example, a uniform resource locator (URL) configured to an end point of the agentic GPT-based interactive ECG analysis platform 102 is provided to the user device 104 for accessing the agentic GPT-based interactive ECG analysis platform 102 and communicating with the agentic GPT-based interactive ECG analysis platform 102. In another example, the agentic GPT-based interactive ECG analysis platform 102 is local to the user device 104 of the user 106.

The user interface 126 is presented on a display of the user device 104 and the user interface 126 includes a chat area 128 that the user 106 uses to provide queries or input messages 130 to the multi-agent query processor 108 (e.g., a copilot engine) of the agentic GPT-based interactive ECG analysis platform 102 and start a chat session with the LLM 120. In some examples, the input message/query 130 provided by the user 106 starts the chat session with the LLM 120. In other examples, the LLM 120 engages with the user 106 by sending the user 106 a proactive message (e.g., a message “Can I help you?”) to start the chat session with the LLM 120. An example input message 130 may include natural language text. The input message can be natural language sentences, questions, requests, code snippets or commands, or any combination of text or code, depending on the domain and the task. One example input message 130 is a question or query. Another example input message 130 is a sentence. Yet another example input message 130 is a portion of a conversation or dialog. In some examples, the input message 130 includes a multistep query.

The multi-agent query processor 108 receives the input message 130 and infers an intent of the input message 130. The multi-agent query processor 108 uses the LLM 120 to process the natural language of the input message 130 to infer the intent of the input message 130. The multi-agent query processor 108 dynamically constructs a prompt with the input message 130, messages from previous chat history, and other conversational context information. Few-shots samples are injected into the prompt to provide examples to let the LLM 120 infer the intent accordingly. The input message 130 is wrapped in the prompt and sent to the LLM 120. The prompt provides contexts for all the capabilities/intents that are supported by the system 100, and the LLM 120 returns the best match given the user input message 130. In some examples, if there is no match, the input message 130 is answered by the LLM 120 itself. A default intent is used when the LLM 120 does not find the best match, and a prompt is used to let the LLM 120 respond with clarification questions to the input message 130.

The multi-agent query processor 108 provides the prompt with the intent and the input message 130 to the LLM 120 via appropriate agents 114. The input prompts are the inputs or queries that a user or a program gives to the LLM 120, in order to elicit a specific response from the LLM 120 in response to the prompt. The LLM 120 uses the intent in generating the response 122 and/or insight 124 to the input message 130. The multi-agent query processor 108 also provides ECG data 118A-118N to the LLM 120 for use in generating the response 122 and/or insight 124 to the input message 130. The multi-agent query processor 108 accesses the data sources 116A-116N and provides the ECG data 118A-118N to the LLM 120.

In some implementations, multi-agent query processor 108 or the agents 114 uses the LLM 120 to rewrite the query from the input message 130 into different alternatives to assist in retrieving the most relevant data from the different data sources 116A-116N. In some implementations, a plurality of relevant results (e.g., 5 to 20 relevant results) are obtained from the ECG data 118A-118N by the LLM 120 for the response 122. The LLM 120 ranks the relevant results and provides a summarization for each relevant result with reasoning on how the relevant results support answering the user's queries.

In some implementations, one or more computing devices (e.g., servers and/or devices) are used to perform the processing of the system 100. The one or more computing devices may include, but are not limited to, server devices, personal computers, a mobile device, such as, a mobile telephone, a smartphone, a PDA, a tablet, or a laptop, and/or a non-mobile device. The features and functionalities discussed herein in connection with the various systems may be implemented on one computing device or across multiple computing devices. For example, the user interface 126 and the agentic GPT-based interactive ECG analysis platform 102 are implemented on the same computing device. Another example includes one or more subcomponents of the user interface 126 and/or the agentic GPT-based interactive ECG analysis platform 102 (e.g., the multi-agent query processor 108, LLM 120, and/or the data sources 116A-116N) are implemented across multiple computing devices. Moreover, in some implementations, one or more subcomponent of the user interface 126 and/or the agentic GPT-based interactive ECG analysis platform 102 may be implemented are processed on different server devices of the same or different cloud computing networks.

In some implementations, each of the components of the system 100 is in communication with each other using any suitable communication technologies. In addition, while the components of the system 100 are shown to be separate, any of the components or subcomponents may be combined into fewer components, such as into a single component, or divided into more components as may serve a particular implementation. In some implementations, the components of the system 100 include hardware, software, or both. For example, the components of the system 100 may include one or more instructions stored on a computer-readable storage medium and executable by processors of one or more computing devices. When executed by the one or more processors, the computer-executable instructions of one or more computing devices can perform one or more methods described herein. In some implementations, the components of the system 100 include hardware, such as a special purpose processing device to perform a certain function or group of functions. In some implementations, the components of the system 100 include a combination of computer-executable instructions and hardware.

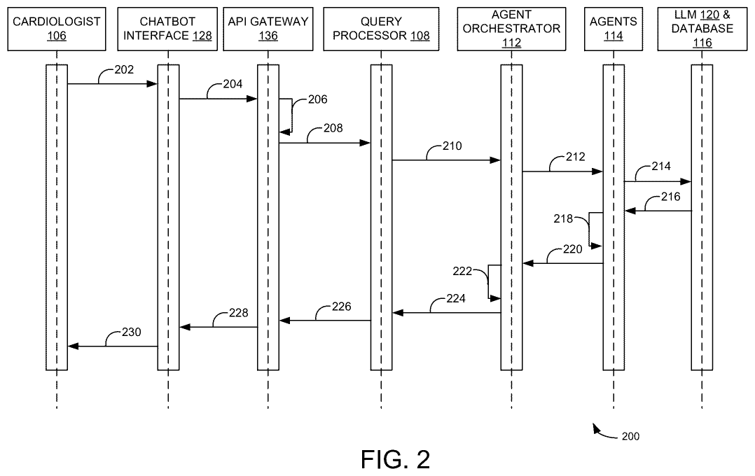

FIG. 2 is an example sequence diagram 200 illustrating a sequence of events to interactively analyze ECG data and generate responses based on user queries. For example, similarly named elements of FIG. 2 may be similar in structure, function, or both to elements described with respect to FIG. 1. Sequence diagram 200 may represent the interactions and the operations involved in interactive ECG analysis and response generation. FIG. 2 illustrates process objects including cardiologist/user 106, chatbot interface 128, API gateway 132, query processor 108, agent orchestrator 112, agents 114, and database 116 along with their respective vertical lines originating from them. The vertical lines of cardiologist/user 106, chatbot interface 128, API gateway 132, query processor 108, agent orchestrator 112, agents 114, and database 116 may represent the processes that may exist simultaneously. The horizontal arrows (e.g., 202, 204, 208, 210, 212, 214, 216, 220, 224, 226, 228, and 230) may represent the data flow steps between the vertical lines originating from their respective process objects (e.g., cardiologist/user 106, chatbot interface 128, API gateway 132, query processor 108, agent orchestrator 112, agents 114, and database 116). Further, activation boxes (e.g., 206, 218, and 222) between the horizontal arrows may represent the process that is being performed in the respective process object.

At 202, the user 106 may enter queries or input messages 130 related to health of a patient, for instance, via chatbot interface 128. At 204, the chatbot interface 128 forwards the query to the API Gateway 132 via an API call. At 206, the API Gateway 132 validates the input message based on predefined rules and data schemas. Upon validating the input message, at 208, the API Gateway 132 forwards the input message to the multi-agent query processor 108.

At 210, the multi-agent query processor 108 determines an intent of the input message, identify data or visualizations required to fulfil the intent, and forward the input message to the agent orchestrator 112. At 212, the agent orchestrator 112 may call relevant agents based on the intent and identified data or visualizations required to fulfil the intent of the input message.

At 214, the relevant agents interact with the database 116 to retrieve the data. At 216, the relevant agents use an LLM or LLMs 120 to fetch contextual data from the database 116 based on the intent and identified data or visualizations required to fulfil the intent. At 218, the relevant agents generate responses based on the fetched contextual data, for instance, using the LLM or LLMs. In other examples, the relevant agents use knowledge representations available to them to fetch contextual data from the database 116 based on the intent and identified data or visualizations required to fulfil the intent. At 220, the relevant agents forward the responses to agent orchestrator 112. At 222, the agent orchestrator 112 consolidates outputs from multiple agents 114 into a unified response, ensuring the information is clear and actionable for the physician. At 224, the agent orchestrator 112 forwards the unified response to the multi-agent query processor 108. At 226, the multi-agent query processor 108 forwards the unified response to the API gateway 132. At 228, the API gateway 132 forwards the unified response to the chatbot interface 128. At 230, the chatbot interface 128 displays the response to the user 106 via a user interface.

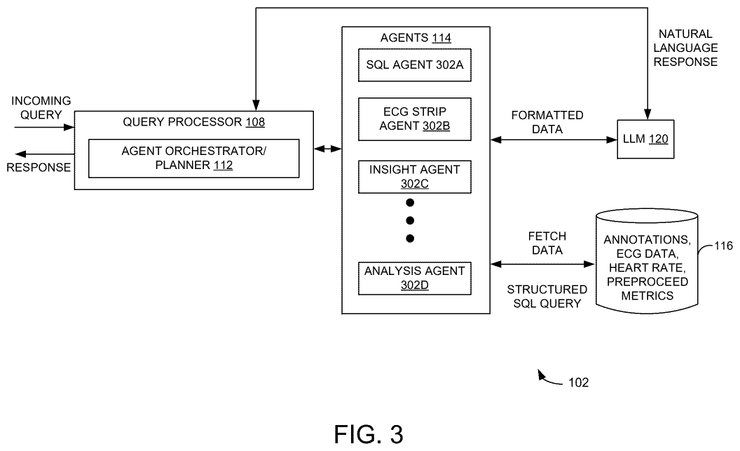

FIG. 3 is a block diagram of the agentic GPT-based interactive ECG analysis platform 102 of FIG. 1, illustrating a data flow between the components. For example, similarly named elements of FIG. 3 may be similar in structure, function, or both to elements described with respect to FIG. 1. The multi-agent query processor 108 may parse the incoming query to determine an intent and identify data or visualizations required to fulfil the intent. Further, multi-agent query processor 108 may send the parsed query to agent orchestrator 112. The agent orchestrator 112 may select agents (e.g., an SQL agent 302A, an ECG strip agent 302B, an insight agent 302C, an analysis agent 302N, and/or so on) based on the identified data or visualizations required to fulfil the intent.

Further, the agent orchestrator/planner 112 coordinates the execution of the selected plurality of agents in a predetermined sequence. Further, the selected agents interact with the database 116 to fetch contextual data from the database 116 based on the intent and identified data or visualizations required to fulfil the intent and the LLM 120. Furthermore, the selected agents generate responses based on the fetched contextual data. The agent orchestrator 112 consolidates outputs from multiple agents into a unified response. The multi-agent query processor 108 displays the unified response on a user interface of the user device.

The agent orchestrator 112 is a powerful tool for implementing sophisticated AI systems comprising multiple specialized agents. The agent orchestrator 112 purpose is to intelligently route user queries to the most appropriate agents while maintaining contextual awareness throughout interactions.

The agent orchestrator 112 follows a specific process for each user request:
1. Request Initiation: The user sends a request to the orchestrator.
2. Classification: The Classifier analyzes the user's request, agent descriptions, and conversation history from all agents for the current user ID and session ID. This comprehensive view allows the classifier to understand ongoing conversations and context across all agents.
   The framework includes two built-in classifier implementations, with one used by default.
   Users can customize many options for these built-in classifiers.
   There's also the option to create your own custom classifier, potentially using models different from those in the built-in implementations.
   The classifier determines the most appropriate agent for:
      A new query requiring a specific agent (e.g., “I want to book a flight” or “What is the base rate interest for a 20-year loan?”)
      A follow-up to a previous interaction, where the user might provide a short answer like “Tell me more”, “Again”, or “12”. In this case, the LLM identifies the last agent that responded and is waiting for this answer.
3. Agent Selection: The Classifier responds with the name of the selected agent.
4. Request Routing: The user's input is sent to the chosen agent.
5. Agent Processing: The selected agent processes the request. It automatically retrieves its own conversation history for the current user ID and session ID. This ensures that each agent maintains its context without access to other agents' conversations.
   The framework provides several built-in agents for common tasks.
   Users have the option to customize a wide range of properties for these built-in agents.
   There's also the flexibility to quickly create your own custom agents for specific needs.
6. Response Generation: The agent generates a response, which may be sent in a standard response mode or via streaming, depending on the agent's capabilities and initialization settings.
7. Conversation Storage: The orchestrator automatically handles saving the user's input and the agent's response into the storage for that specific user ID and session ID. This step is crucial for maintaining context and enabling coherent multi-turn conversations. Key points about storage:
   The framework provides two built-in storage options: in-memory and DynamoDB.
   You have the flexibility to quickly create and implement your own custom storage solution and pass it to the orchestrator.
   Conversation saving can be disabled for individual agents that don't require follow-up interactions.
   The number of messages kept in the history can be configured for each agent.
8. Response Delivery: The orchestrator delivers the agent's response back to the user.

This process ensures that each request is handled by the most appropriate agent while maintaining context across the entire conversation. The classifier has a global view of all agent conversations, while individual agents only have access to their own conversation history. This architecture allows for intelligent routing and context-aware responses while maintaining separation between agent functionalities.

The orchestrator's automatic handling of conversation saving and fetching, combined with flexible storage options, provides a powerful and customizable system for managing conversation context in multi-agent scenarios. The ability to customize or replace classifiers and agents offers further flexibility to tailor the system to specific needs.

The Multi-Agent Orchestrator framework empowers you to leverage multiple agents for handling diverse tasks. In the framework context, an agent can be any of the following (or a combination of one or more):
LLMs (through Amazon Bedrock or any other cloud-hosted or on-premises LLM)
API calls
AWS Lambda functions
Local processing
Amazon Lex Bot
Amazon Bedrock Agent
Any other specific task or process

This flexible architecture allows you to incorporate as many agents as your application requires, and combine them in ways that best suit your needs. Each agent needs a name and a description (plus other properties specific to the type of agent you use). The agent description plays a crucial role in the orchestration process. It should be detailed and comprehensive, as the orchestrator relies on this description, along with the current user input and the conversation history of all agents, to determine the most appropriate routing for each request.

While the framework's flexibility is a strength, it's important to be mindful of potential overlaps between agents, which could lead to incorrect routing. To help you analyze and prevent such overlaps, we recommend reviewing our agent overlap analysis section for a deeper understanding.

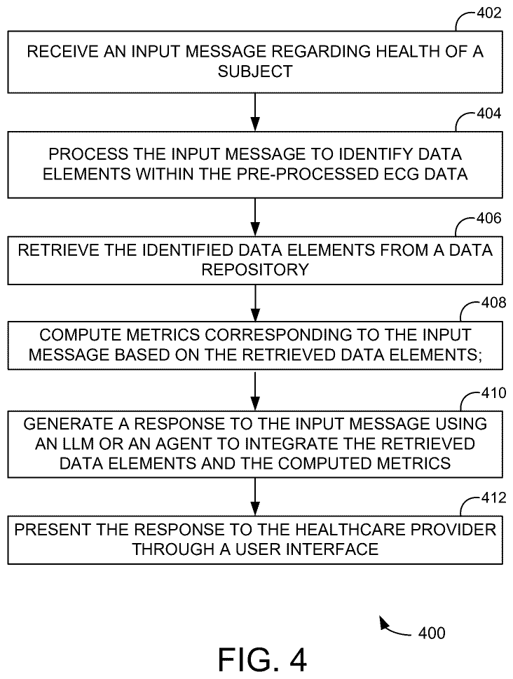

FIG. 4 is a flow diagram illustrating an example method 400 for analyzing pre-processed ECG data. Example method 400 depicted in FIG. 4 represents generalized illustrations, and other processes may be added, or existing processes may be removed, modified, or rearranged without departing from the scope and spirit of the present application. In addition, method 400 may represent instructions stored on a computer-readable storage medium that, when executed, may cause a processor to respond, to perform actions, to change states, and/or to make decisions. Alternatively, method 400 may represent functions and/or actions performed by functionally equivalent circuits like analog circuits, digital signal processing circuits, application specific integrated circuits (ASICs), or other hardware components associated with the system. Furthermore, the flow chart is not intended to limit the implementation of the present application, but the flow chart illustrates functional information to design/fabricate circuits, generate computer-readable instructions, or use a combination of hardware and computer-readable instructions to perform the illustrated processes.

At 402, an input message regarding health of a subject is received. The input message is received via a chatbot interface, for instance. In an example, the input message specifies a time period for analysis. In another example, the input message may include a comparison of data across multiple subjects. In another example, the input message may include a comparison of data across multiple studies for a single subject. In some examples, the method further includes a prompt engineering step to refine the input message for interpretation by the multi-agent orchestrator.

At 404, the input message is processed using a multi-agent orchestrator to identify data elements from the pre-processed ECG data. The pre-processed ECG data is associated with historical data, real-time data, or both derived from a plurality of electrographic recorders. The pre-processed ECG data may include ECG measurements extracted or derived from raw ECG signals and annotations of cardiac events. For example, the ECG measurements comprise PR interval, PR segment, QRS complex duration, heart rate variability, R-R intervals, QT intervals, ST interval, ST segment, and data for assessing the cardiac events, or any combination thereof. Also, example cardiac events may include arrhythmias such as Atrial Fibrillation, Atrial Flutter, Pause, Tachycardia, Bradycardia, Couplet, Triplet, Bigeminy, Trigeminy, Quadrigeminy, Premature Atrial Contraction (PAC), Atrial Tachycardia, Premature Ventricular Contraction (PVC), Idioventricular rhythm, Ventricular Tachycardia, Second degree MOBITZ-1 AV Block, Second degree MOBITZ-2 AV Block, Third degree AV Block, High degree AV Block and any cardiovascular irregularities.

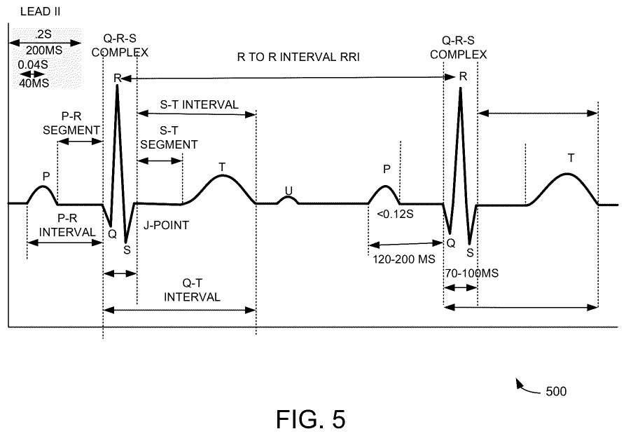

An example ECG waveform 500, depicting the ECG measurements is shown in FIG. 5. As shown in in FIG. 5, the ECG waveform 500 may depict various ECG measurements such as a P wave, a QRS complex, a T wave, an ST segment, a PR interval, and a QT interval.

The P wave, a component of an electrocardiogram (ECG), represents the electrical activity associated with atrial depolarization, which initiates the contraction of the atria. A normal P wave typically has a duration of no more than 0.12 seconds (less than 3 small squares on standard ECG paper) and an amplitude (height) of no more than 3 millimeters. It should also exhibit a smooth, rounded shape without any notching or peaking.

The QRS complex on the ECG represents the electrical activity associated with ventricular depolarization, the process that initiates the contraction of the heart's lower chambers (the ventricles). A normal QRS complex typically has a duration of no longer than 0.10 seconds and an amplitude of at least 5 mm in lead II or 9 mm in leads V3 and V4. R waves are typically deflected positively, while Q and S waves are typically deflected negatively. These characteristics are essential for interpreting the health of the heart's electrical conduction system.

The T wave represents the electrical activity associated with ventricular repolarization, the process where the ventricles of the heart return to their resting state after contraction. A normal T wave typically has an amplitude of no more than 5 mm in standard leads and 10 mm in precordial leads. It is characterized by a rounded and asymmetrical shape.

The ST segment represents the early phase of ventricular repolarization, the period between ventricular depolarization and repolarization. Normally, the ST segment is not depressed more than 0.5 millimeters below the baseline. In some leads, slight elevation of the ST segment may be observed, typically not exceeding 1 millimeter.

The PR interval represents the time it takes for the electrical impulse to travel from the atria (upper chambers of the heart) to the ventricles (lower chambers). A normal PR interval typically ranges from 0.12 to 0.20 seconds, indicating the time required for the electrical signal to pass through the atrioventricular (AV) node, which acts as a gatekeeper between the atria and ventricles.

The QT interval, measured from the start of the QRS complex to the end of the T wave, reflects ventricular depolarization and repolarization. Normally, it's less than half the R-R interval and varies with heart rate. Prolonged QT, exceeding 0.46 seconds in women and 0.45 seconds in men (corrected for heart rate), can be inherited or acquired (e.g., by medications). This condition increases the risk of dangerous heart rhythms.

Referring back to FIG. 4, in an example, processing the input message may include:
performing semantic analysis on the received input message to determine an intent of the input message and to identify data or visualizations required to fulfil the intent,
selecting an agent based on the identified data or visualizations required to fulfil the intent of the input message, and
receiving an output from the selected agent, the output comprising the identified data elements and/or the computed metrics.

In another example, processing the input message may include:
performing semantic analysis on the received input message to determine an intent of the input message and to identify data or visualizations required to fulfil the intent,
selecting a plurality of agents based on the identified data or visualizations required to fulfil the intent of the input message,
coordinating the execution of the selected plurality of agents in a predetermined sequence,
receiving outputs from each of the selected plurality of agents, the outputs comprising the identified data elements and/or the computed metrics, and
aggregating the received outputs from the selected plurality of agents into a unified response.

At 406, the identified data elements may be retrieved from a data repository. The data repository may include the pre-processed ECG data and the raw ECG signals associated with real-time data and tagged with subject's metadata. At 408, metrics corresponding to the input message may be computed based on the retrieved data elements. In an example, the retrieved data elements and/or the computed metrics may include visual representations of ECG data for specific events.

At 410, a response to the input message may be generated using an LLM or at least one agent to integrate the retrieved data elements and the computed metrics. The response may include textual information, graphical representation, or both. At 412, the response may be presented to the healthcare provider through a user interface. The method further includes monitoring real-time pre-processed ECG data stream to detect a critical event, and generating an alert including an actionable insight based on the critical event.

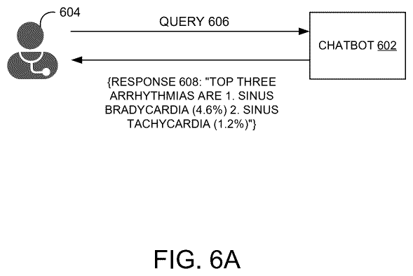

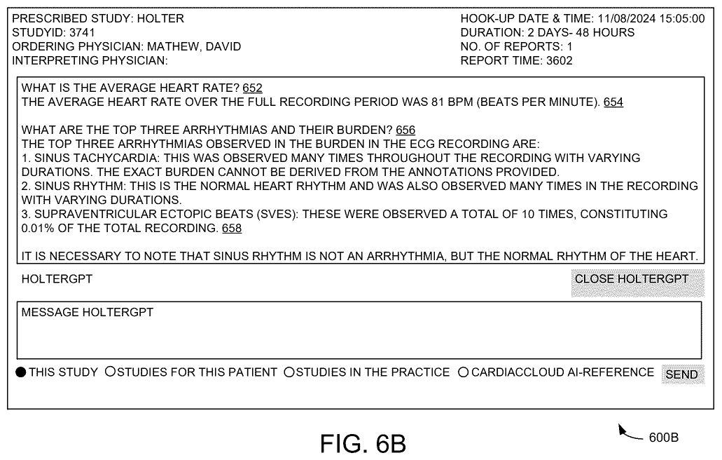

FIG. 6A is a schematic diagram, depicting an example interaction between a physician 604 and a chatbot 602. An arrow indicates that the physician 604 is sending a query 606 to the chatbot 602. Further, the chatbot 602 responds to the query 606 with a message 606 indicating “TOP THREE ARRHYTHMIAS” as 1. Sinus Bradycardia (4.6%), 2. Sinus Tachycardia (1.2%).

FIG. 6B is an example user interface 600B, depicting an example interaction between a physician and a chatbot. Particularly, FIG. 6B depicts a physician's query 652 “What is the average heart rate?” and associated response 654 “THE Average heart rate over the full recording period was 81 bpm (beats per minute)” generated by the chatbot. Similarly, FIG. 6B also depicts a physician's query 656 and associated response 658 generated by the chatbot.

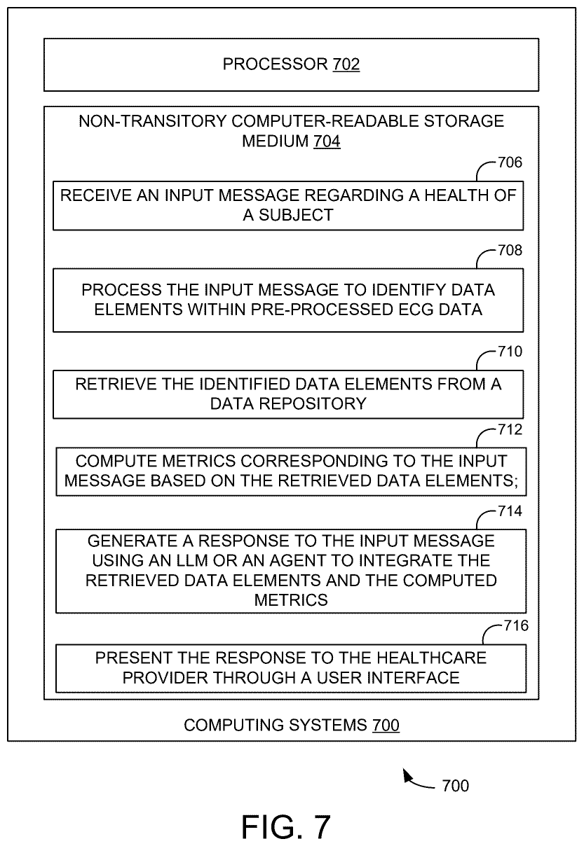

FIG. 7 is a block diagram of an example computing device 700 including non-transitory computer-readable storage medium storing instructions to analyze pre-processed ECG data. Computing device 700 may include a processor 702 and computer-readable storage medium 704 communicatively coupled through a system bus. Processor 702 may be any type of central processing unit (CPU), microprocessor, or processing logic that interprets and executes computer-readable instructions stored in computer-readable storage medium 704. Computer-readable storage medium 704 may be a random-access memory (RAM) or another type of dynamic storage device that may store information and computer-readable instructions that may be executed by processor 702. For example, computer-readable storage medium 704 may be synchronous DRAM (SDRAM), double data rate (DDR), Rambus® DRAM (RDRAM), Rambus® RAM, etc., or storage memory media such as a floppy disk, a hard disk, a CD-ROM, a DVD, a pen drive, and the like. In an example, computer-readable storage medium 704 may be a non-transitory computer-readable medium. In an example, computer-readable storage medium 704 may be remote but accessible to computing device 700.

Computer-readable storage medium 704 may store instructions 706, 708, 710, 712, 714, and 716. Instructions 706 may be executed by processor 702 to receive an input message regarding health of a subject. Instructions 708 may be executed by processor 702 to process the input message using a multi-agent orchestrator to identify data elements from pre-processed ECG data.

Instructions 710 may be executed by processor 702 to retrieve the identified data elements from a data repository. Instructions 712 may be executed by processor 702 to compute metrics corresponding to the input message based on the retrieved data elements. Instructions 714 may be executed by processor 702 to generate a response to the input message using an LLM or at least one agent to by integrate the retrieved data elements and the computed metrics. Instructions 716 may be executed by processor 702 to present the response to the healthcare provider through a user interface.

The above-described examples are for the purpose of illustration. Although the above examples have been described in conjunction with example implementations thereof, numerous modifications may be possible without materially departing from the teachings of the subject matter described herein. Other substitutions, modifications, and changes may be made without departing from the spirit of the subject matter. Also, the features disclosed in this specification (including any accompanying claims, abstract, and drawings), and any method or process so disclosed, may be combined in any combination, except combinations where some of such features are mutually exclusive.

The terms “include,” “have,” and variations thereof, as used herein, have the same meaning as the term “comprise” or appropriate variation thereof. Furthermore, the term “based on”, as used herein, means “based at least in part on.” Thus, a feature that is described as based on some stimulus can be based on the stimulus or a combination of stimuli including the stimulus. In addition, the terms “first” and “second” are used to identify individual elements and may not meant to designate an order or number of those elements.

The present description has been shown and described with reference to the foregoing examples. It is understood, however, that other forms, details, and examples can be made without departing from the spirit and scope of the present subject matter that is defined in the following claims.

What is claimed is:

1. A method for analyzing pre-processed electrocardiographic (ECG) data, comprising:
receiving an input message regarding health of a subject;
processing the input message using a multi-agent orchestrator to identify data elements from the pre-processed ECG data, wherein the pre-processed ECG data is associated with historical data, real-time data, or both derived from a plurality of electrographic recorders, and wherein the pre-processed ECG data includes ECG measurements extracted or derived from raw ECG signals and annotations of cardiac events;
retrieving the identified data elements from a data repository;
computing metrics corresponding to the input message based on the retrieved data elements;
generating a response to the input message using a large language model (LLM) or at least one agent to integrate the retrieved data elements and the computed metrics; and
presenting the response to the healthcare provider through a user interface.

2. The method of claim 1, wherein processing the input message comprises:
performing semantic analysis on the received input message to determine an intent of the input message and to identify data or visualizations required to fulfil the intent;
selecting an agent based on the identified data or visualizations required to fulfil the intent of the input message; and
receiving an output from the selected agent, the output comprising the identified data elements and/or the computed metrics.

3. The method of claim 1, wherein processing the input message comprises:
performing semantic analysis on the received input message to determine an intent of the input message and to identify data or visualizations required to fulfil the intent;
selecting a plurality of agents based on the identified data or visualizations required to fulfil the intent of the input message;
coordinating the execution of the selected plurality of agents in a predetermined sequence;
receiving outputs from each of the selected plurality of agents, the outputs comprising the identified data elements and/or the computed metrics; and
aggregating the received outputs from the selected plurality of agents into a unified response.

4. The method of claim 1, further comprising:
monitoring real-time pre-processed ECG data stream to detect a critical event; and
generating an alert including an actionable insight based on the critical event.

5. The method of claim 1, wherein the ECG measurements comprise PR interval, PR segment, QRS complex duration, heart rate variability, R-R intervals, QT intervals, ST interval, ST segment, and data for assessing the cardiac events, or any combination thereof.

6. The method of claim 1, wherein the cardiac events comprise arrhythmias and/or any cardiovascular irregularities.

7. The method of claim 1, wherein the input message specifies a time period for analysis, a comparison of data across multiple subjects, a comparison of data across multiple studies for a single subject, or any combination thereof.

8. The method of claim 1, wherein the response comprises insights associated with the input message.

9. The method of claim 1, wherein the retrieved data elements and/or the computed metrics include visual representations of ECG data for specific events.

10. The method of claim 1, wherein the response includes textual information, graphical representation, or both.

11. The method of claim 1, wherein the input message is received via a chatbot interface.

12. The method of claim 1, wherein the data repository comprises the pre-processed ECG data and the raw ECG signals associated with the historical data, real-time data, or both and tagged with subject's metadata.

13. The method of claim 1, further comprising a prompt engineering step to refine the input message for interpretation by the multi-agent orchestrator.

14. A system for interactive electrocardiographic (ECG) monitoring, comprising:
a data repository storing pre-processed ECG data, wherein the pre-processed ECG data is associated with historical data, real-time data, or both derived from a plurality of electrographic recorders, and wherein the pre-processed ECG data includes ECG measurements extracted or derived from raw ECG signals and annotations of cardiac events;
a multi-agent query processor configured to interact with the data repository and the LLM to:
receive and process an input message related to health of a subject;
retrieve relevant data elements from the pre-processed ECG data, raw ECG signals, or both based on the processed input message;
compute metrics corresponding to the input message based on the retrieved data elements; and
generate a response to the input message using a large language model (LLM) or at least one agent to integrate the retrieved data elements and the computed metrics; and
a user interface to present the response generated by the LLM to a healthcare provider.

15. The system of claim 14, wherein the multi-agent query processor comprises:
a SQL agent to retrieve structured ECG data from the data repository;
an insights agent to derive and compute contextual insights related to the input message;
an ECG strip agent to generate visual representations of the retrieved ECG data; and
a tool-calling planner for coordinating the execution of the SQL, insights, and ECG strip agents.

16. The system of claim 14, further comprising:
a validation unit to validate the input message based on predefined rules and data schemas prior to processing the input message.

17. A non-transitory computer readable storage medium having instructions executable by a processor of a computing device to:
receive an input message regarding health of a subject;
process the input message using a multi-agent orchestrator to identify data elements from pre-processed electrocardiographic (ECG) data, wherein the pre-processed ECG data is associated with historical data, real-time data, or both derived from a plurality of electrographic recorders, and wherein pre-processed ECG data includes ECG measurements extracted or derived from raw ECG signals and annotations of cardiac events;
retrieve the identified data elements from a data repository;
computing metrics corresponding to the input message based on the retrieved data elements;
generate a response to the input message using a large language model (LLM) or at least one agent to integrate the retrieved data elements and the computed metrics; and
present the response to the healthcare provider through a user interface.

18. The non-transitory computer readable storage medium of claim 17, wherein instructions to process the input message comprise instructions to:
perform semantic analysis on the received input message to determine an intent of the input message and to identify data or visualizations required to fulfil the intent;
select an agent based on the identified data or visualizations required to fulfil the intent of the input message; and
receive an output from the selected agent, the output comprising the identified data elements and/or the computed metrics.

19. The non-transitory computer readable storage medium of claim 17, wherein instructions to process the input message comprise instructions to:
perform semantic analysis on the received input message to determine an intent of the input message and to identify data or visualizations required to fulfil the intent;
select a plurality of agents based on the identified data or visualizations required to fulfil the intent of the input message;
coordinate the execution of the selected plurality of agents in a predetermined sequence;
receive outputs from each of the selected plurality of agents, the outputs comprising the identified data elements and/or the computed metrics; and
aggregate the received outputs from the selected plurality of agents into a unified response.

20. The non-transitory computer readable storage medium of claim 17, wherein the ECG measurements comprise PR interval, PR segment, QRS complex duration, heart rate variability, R-R intervals, QT intervals, ST interval, ST segment, and data for assessing the cardiac events, or any combination thereof.
